(19) United States
(12) Patent Application Publication
(10) Pub. No.: US 2021/0038805 A1
(43) Pub. Date: Feb. 11, 2021

(54) ELECTRONIC PATCH FOR TRANSDERMAL DELIVERY OF MEDICAL COMPOSITIONS

(71) Applicant: RYAH Medtech, Inc., Brooklyn, NY (US)

(72) Inventor: Boris Goldstein, New York, NY (US)

(21) Appl. No.: 16/938,663

(22) Filed: Jul. 24, 2020

Related U.S. Application Data
(60) Provisional application No. 62/879,283, filed on Jul. 26, 2019.

(51) Int. Cl. A61M 5/142; A61M 5/172

(52) U.S. Cl. CPC A61M 5/14248; A61M 5/172; A61M 2205/3633; A61M 2207/10; A61M 2205/8206; A61M 2205/3553; A61M 2205/583; A61M 2205/50

(57) ABSTRACT

This invention provides an electronic patch for transdermal medicine delivery. The electronic patch has a medicine reservoir and an electronic controller. The electronic controller is capable of changing operation parameters such as temperature and heating intervals to adapt with the medicine and adjust delivery rate. The electronic patch can be controlled remotely and can collect data to communicate with other computing processors. Data collected may be stored and used to provide treatment for the user and can also be used for study and research.

### CROSS REFERENCE TO RELATED APPLICATIONS

This application claims the benefit of U.S. Provisional Application No. 62/879,283, filed Jul. 26, 2019. Each of the above-referenced patent applications is incorporated by reference in its entirety.

### BACKGROUND OF THE INVENTION

### Field of the Invention

This invention concerns a patch for transdermal delivery of medicament. The rate of delivery of the medicament may be controlled, adjusted, and recorded by electronic means.

### Description of the Related Technology

Transdermal delivery of medicine has been known throughout history in many cultures. The first Food and Drug Administration-approved medical patch was approved in 1979, which administered scopolamine for motion sickness and is still popular today. Other patches are now commonly used, such as nicotine patch, fentanyl patch, buprenorphine patch, hormonal patches, as well as an array of other medical patches delivering many different medicines.

Transdermal delivery of medicine has many advantages, including a controlled release of medicine and provides a safer route to receive an active ingredient than a traditional administration route. An example is nicotine patch for smoking cessation, where receiving nicotine through a patch is safer than through inhalation of smoke. The disadvantage is the fact that the skin is an effective barrier and thus far only smaller molecules may be delivered through the skin.

Cannabis as medicine has received wide recognition and acceptance in the United States and in many other countries recently. Various cannabis delivery mechanisms are available. Oral administration, inhalation, suppositories, and transdermal patches are popular medical cannabis delivery methods.

Smoking, vaporization, and other similar methods provide fast absorption but short plasma concentration profile of cannabinoids. Some patient populations may have other concerns when it comes to the inhalation delivery route, including children and patients with concomitant lung diseases. Oral ingestion of cannabis may provide convenient and longer lasting effects, but results in very low bioavailability of the drug and takes a long time for effects to arrive. Medicinal cannabis delivery should be suited for the application in terms of pharmacokinetics profile. It should also be safe, easy to use, seamless, repeatable and controllable. A constant drug plasma concentration profile and a discrete method of delivery are also desirable. Transdermal cannabis delivery offers an opportunity in becoming such a delivery route.

Transdermal delivery of active ingredients has as the main barrier the limitation in the molecular size of the active ingredients. Nicotine, for example, has a molecular size of 160 kDa (kilo Dalton) while cannabinoids—active ingredients in cannabis—have molecular sizes of about 300 kDa, which is close to the upper limit of molecular size deliverable transdermally.

Currently, there are simple cannabis patches including THC patches, CBD patches, CBN patches, CBG patches, and combination of cannabinoid patches. Unfortunately, such simple patches can only provide low dosage with relatively high variability. The same challenge exists for other medical patches aiming at delivering higher molecular weight compositions. Hence, acceleration methods need to be used to increase skin permeation of the drug. These methods may include heating, chemical permeability enhancers, iontophoresis, sonophoresis, skin preparation, microneedle array and other methods.

There remains a need for effective delivery of medical molecules of higher molecular weight transdermally, where other administration routes may be undesirable. At the same time, delivery rate of medicine through the transdermal route has not been adequately tracked or studied to enhance efficacy of delivery by this method. This invention seeks to solve these problems.

### SUMMARY

This invention discloses an electronic patch having a medicament compartment and an electronic controller. The electronic controller controls and exerts heat onto the medicament compartment, thereby controlling delivery rate and operation parameters. The medicament compartment may be disposable and replaced by another medicament compartment. Data concerning operation parameters may be communicated to other computing processors, stored, aggregated, and studied.

This invention provides an electronic transdermal patch for delivery of medicament, comprising:
a medicament compartment to contain a medicament, comprising:
   a reservoir configured to hold a medicament, the reservoir having on one end a first protective film layer and on the other end a rate controlling membrane, the first protective film payer and the rate controlling membrane situated approximately opposing each other and separated by the height of the reservoir;
   an adhesive pad attached to the reservoir at the end where the rate controlling membrane resides, the adhesive pad being in contact with the reservoir at the perimeter of the reservoir; and
   a second protective film layer in physical contact with the adhesive pad on the opposite side from the rate controlling membrane;
an electronic controller in contact with and operatively connected to the reservoir, the electronic controller comprising:
   a thermal conductive layer configured to conduct thermal energy to the medicament compartment, the thermal conductive layer being in physical contact with and operatively connected to the first protective film layer;
   a flexible circuit board having a heating element and a thermistor;
   a motherboard having electronic circuits and electronic components attached to the motherboard, the motherboard operatively connected to the flexible circuit board; and
   a thermal isolation layer situated between the flexible circuit board and the motherboard, the thermal isolation layer configured to substantially reduce thermal energy conduction from the flexible circuit board;
   a battery operatively connected to the motherboard; and
a housing to house the electronic controller components;
wherein the motherboard comprises a microcontroller, and
wherein the medicament compartment and the electronic controller are operatively connected to each other.

This invention provides an electronic patch as above, wherein the electronic controller is configured to collect data from the electronic patch.

This invention provides an electronic patch as above, wherein the data collected comprise temperature during heating episodes of the medicament compartment, length of the heating episodes, frequency of the heating episodes, and types of medicament.

This invention provides an electronic patch as above, wherein the electronic controller is configured to wirelessly communicate with another computing processor.

This invention provides an electronic patch as above, wherein the medicament compartment further comprises an electronic information storage means having information concerning the medicament contained inside.

This invention provides an electronic patch as above, wherein the electronic controller is configured to wirelessly receive commands from a computing processor and execute the commands received.

This invention provides an electronic patch as above, wherein the medicament compartment is capable of being removed from electronic controller and replaced by another, similar medicament compartment.

This invention provides an electronic patch as above, wherein the medicament compartment is produced by three-dimensional printing.

This invention provides an electronic patch as above, wherein the electronic controller is configured to transmit thermal energy to the medicament compartment in intervals.

This invention provides an electronic patch as above, further comprising a medicament residing inside the reservoir.

This invention provides an electronic patch as above, further comprising a foam tape situated at the top of the thermal isolation layer.

This invention provides an electronic patch as above, wherein the foam tape comprises two adhesive layers.

This invention provides an electronic patch as above, further comprising a Light Emitting Diode display to indicate operating status of the electronic patch.

This invention provides an electronic patch as above, further comprising a computer programming product operable on a remote computing article and providing an electronic control interface capable of interacting with the electronic patch.

This invention provides an electronic patch as above, wherein the computer programming product is configured to send operating commands to the electronic patch, receiving data from the electronic patch, storing and aggregating received data, and conducting machine learning based in the received data.

This invention provides an electronic patch as above, further comprising an accelerometer configured to detect movement of the user and communicate collected data to the motherboard, wherein the motherboard uses collected data to adjust the electronic patch's operation.

This invention provides an electronic patch as above, further comprising a placement detection button to detect the body part where the electronic patch is placed, wherein the detection button is configured to communicate this information to the motherboard and wherein the motherboard uses collected data to adjust the electronic patch's operation.

This invention provides a method to deliver medicine transdermally, comprising the steps of:
placing the electronic transdermal patch on a user's skin at an administration site, the electronic patch comprising:
   a medicament compartment to contain a medicament, comprising:
      a reservoir configured to hold a medicament, the reservoir having on one end a first protective film layer and on the other end a rate controlling membrane, the first protective film payer and the rate controlling membrane situated approximately opposing each other and separated by the height of the reservoir;
      an adhesive pad attached to the reservoir at the end where the rate controlling membrane resides, the adhesive pad being in contact with the reservoir at the perimeter of the reservoir; and
      a second protective film layer in physical contact with the adhesive pad on the opposite side from the rate controlling membrane;
   an electronic controller in contact with and operatively connected to the reservoir, the electronic controller comprising:
      a thermal conductive layer configured to conduct thermal energy to the medicament compartment, the thermal conductive layer being in physical contact with and operatively connected to the first protective film layer;
      a flexible circuit board having a heating element and a thermistor;
      a motherboard having electronic circuits and electronic components attached to the motherboard, the motherboard operatively connected to the flexible circuit board;
      a thermal isolation layer situated between the flexible circuit board and the motherboard, the thermal isolation layer configured to substantially reduce thermal energy conduction from the flexible circuit board;
      a foam tape situated at the top of the thermal isolation layer;
      a battery operatively connected to the motherboard;
   a housing to house the electronic controller components;
   a medicament residing inside the reservoir; and
   a computer programming product operable on a remote computing article and providing an electronic control interface capable of interacting with the electronic patch;
   wherein the motherboard comprises a microcontroller,
   wherein the medicament compartment and the electronic controller are operatively connected to each other, and
   wherein the computer programming product is configured to send operating commands to the electronic patch, receiving data from the electronic patch, storing and aggregating received data, and conducting machine learning based in the received data;
opening the electronic control interface on the remote computing article;
setting operating parameters for the electronic transdermal patch; and
starting the operating procedure for the electronic transdermal patch.

This invention provides a method to deliver medicine transdermally as above, wherein the operating parameters are temperature, duration of heating cycles, and frequency of heating cycles.

This invention provides a method to deliver medicine transdermally as above, further comprising the steps of:
collecting data from the electronic patch's operation;
transmitting collected data to a remote computing processor;
storing collected data in a computing article; and
analyzing stored data to make medical care decision.

This invention provides a method to conduct medical studies, comprising the steps of:
placing the electronic transdermal patch as above on a user's skin at an administration site;
opening the electronic control interface on the remote computing processor;
setting operating parameters for the electronic transdermal patch;
starting the operating procedure for the electronic transdermal patch;
collecting data from the heating cycles;
transmitting collected data to a remote computing processor;
storing collected data in a computing article;
repeating the above steps with various electronic patches and various users for a finite time;
analyzing stored data to make medical care decision; and
aggregating data from different users and conducting research and study based on the aggregated data.

### Abbreviations

CBD: cannabidiol

CBG: cannabigerol

CBN: cannabinol

FCB: Flexible Circuit Board

FDA: Food and Drug Administration

IC: Integrated Circuit

kDa: kilo Dalton

LE: Low Energy

LED: Light Emitting Diode

NFC: Near Field Communication

FCB: Flexible Circuit Board

PCB: Printed Circuit Board

QR: Quick Response

RGB: Red Green Blue

SWD: Serial Wire Debug

THC: tetrahydrocannabinol

### BRIEF DESCRIPTION OF THE DRAWINGS

FIG. 1 is an exploded view of the electronic patch medicament compartment.

FIG. 2 is the top view of the second protective film layer.

FIG. 3 is the top view of the adhesive pad.

FIG. 4 is the top view of the rate controlling membrane.

FIG. 5 is the top view of the reservoir retainer layer.

FIG. 6 is the cross-sectional view of the electronic patch taken along the X-X axis.

FIG. 7 is the exploded view of the heating block within the electronic controller.

FIG. 8 is the top view of the thermal isolation film layer.

FIG. 9 is the top view of the thermal conductive film layer.

FIG. 10 is the top view of the foam tape.

FIG. 11 is the cross-sectional, exploded view of the electronic patch' medicament reservoir and heating block taken along the Y-Y axis.

FIG. 12 is the top view of the motherboard and the battery.

FIG. 13 is the top view of the motherboard with drill holes.

FIG. 14A is a perspective view of the medicament compartment detached from the electronic controller.

FIG. 14B is the top view of the medicament compartment.

FIG. 14C is the cross sectional view along the Z-Z axis of the medicament compartment.

FIG. 15A is a perspective view of the electronic controller detached from the medicament compartment.

FIG. 15B is the top view of the electronic controller detached from the medicament compartment.

FIG. 15C is the right side view of the electronic controller detached from the medicament compartment.

FIG. 16A is a perspective view of the electronic patch completely assembled.

FIG. 16B is the top view of the electronic patch completely assembled.

FIG. 16C is the right side view of the electronic patch completely assembled.

FIG. 17 is the operating chart of the electronic components within the electronic controller.

### DETAILED DESCRIPTION OF CERTAIN INVENTIVE EMBODIMENTS

The detailed description set forth below in connection with the appended drawings is intended as a description of exemplary embodiments of the invention and is not intended to represent the only embodiments in which the invention may be practiced. The term “exemplary” used throughout this description means “serving as an example, instance, or illustration,” and should not necessarily be construed as preferred or advantageous over other exemplary embodiments. The detailed description includes specific details for the purpose of providing a thorough understanding of the exemplary embodiments of the invention. In some instances, some devices are shown in block diagram form.

The drawings presented herein are not to scale. It is to be understood that different dimensions are contemplated for the electronic patch according to embodiments.

As used herein, the verb “to comprise” in this description, claims, and other conjugations are used in its non-limiting sense to mean those items following the word are included, but items not specifically mentioned are not excluded.

As used herein, the term “thermal conductive” means the ability to conduct heat from one physical medium or object to another at a high rate. For example, a thermal conductive layer can receive heat from a source and conduct heat from that source to another object.

As used herein, the term “thermal isolation” means the ability to prevent heat from transferring from one physical medium or object to another, such that any heat conduction is at a low rate. For example, a thermal isolation layer can receive heat from a source and prevent heat from being transferred from that source to another object.

As used herein, the term “transdermal” or “transdermally” means the crossing of matter or energy through the skin of mammals.

As used herein, the term “heating block” means a group of devices or things that work together to provide heat and/or conduct heat.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more”.

Embodiments of this application relate to a patch for transdermal delivery of medicine in a mammal, preferably a human being. The electronic patch has an electronic controller component capable of controlling operating parameters to control the delivery of the medicament in certain manners. The electronic patch also has a medicament compartment that may be customized and removed from the electronic controller. Operating data may be collected and communicated to remote computing processors. The electronic patch may be controlled by a remote computing program product, such as an application on a mobile device or other software on other computing articles.

In embodiments, the medicament compartment may operate to hold the medicament and allow its transdermal transport. The medicament compartment may be configured to allow controlled delivery rate of the medicament, wherein the medicament is heated for certain intervals at certain temperatures. Certain other operating parameters may also be exerted on the medicament compartment. The medicament compartment may comprise, generally, a reservoir defined by a surrounding wall with a protective film layer at the top, a retainer layer which may be made from medical nonwoven fabric resting on a rate controlling membrane where the medicament may pass through during operation, an adhesive pad attached to the medicament reservoir underneath the rate controlling membrane, and a second protective film layer to protect the adhesive pad.

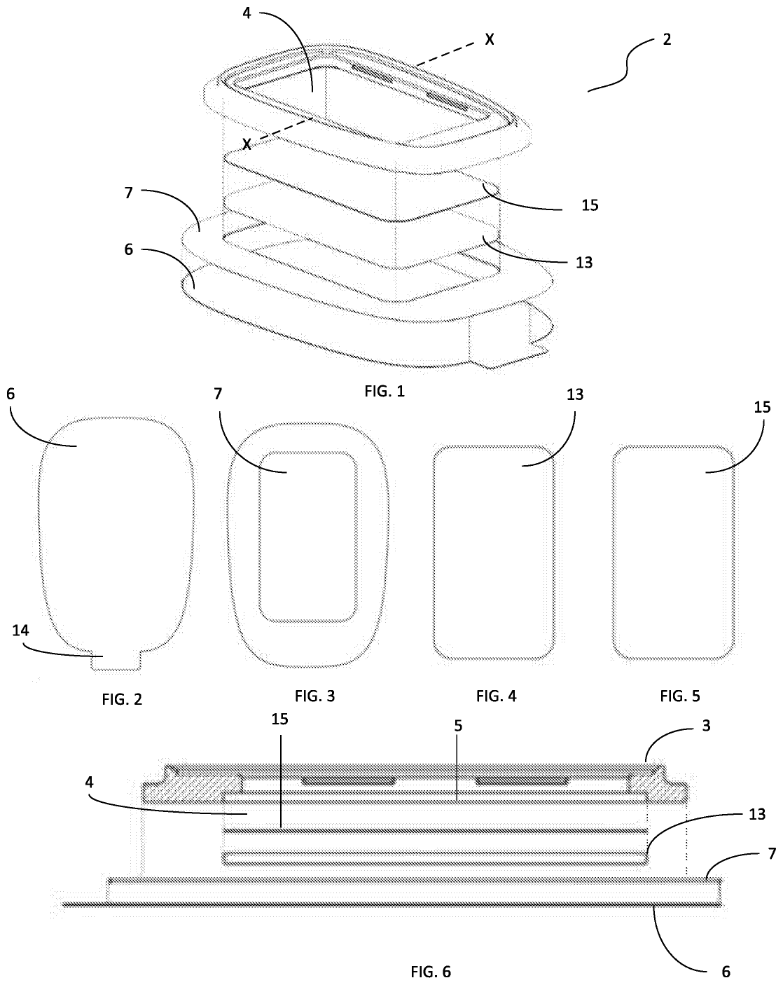

FIG. 1 is the exploded view of the medicament compartment 2. At the bottom may be the second protective film layer 6, which may be removable. Prior to use, the second protective film layer 6 may be attached to the adhesive pad 7 to protect the adhesive pad 7 and the rate controlling membrane 13 from contaminants and exposure to the environment. The second protective film layer 6 may be removed to expose the adhesive pad 7, which may be attached to the subject's skin upon use.

In embodiments, attached to the adhesive pad 7 on the opposite side of the second protective film layer 6 may be a rate controlling membrane 13, where the medicament may pass through when the electronic patch 1 is worn by a user. The rate controlling membrane 13 may differ from one electronic patch 1 to another and may be chosen according to the medicament and/or release rate desired. By way of example, suitable materials for the rate controlling membrane may be ethylene vinyl acetate membrane film, such as 3M CoTran Ethylene Vinyl Acetate Membrane Film, 9702 or 3M CoTran Ethylene Vinyl Acetate Membrane Film, 9712 by 3M; or fluoropolymer coated polyester film, such as 3M Scotchpak 1022 Release Liner Fluoropolymer Coated Polyester Film.

In embodiments, above the rate controlling membrane 13 may be the medicament reservoir 4. At the bottom of the reservoir 4 may be a reservoir retainer layer 15 made of medical nonwoven fabric layer in contact with the rate controlling membrane 13, whereon the medicament may be deposited. At the top of the medicament reservoir 4 may be the first protective film layer 5 (shown in FIG. 6), which may protect the medicament in the medicament reservoir 4 from contact with the electronic controller 3. The medicament reservoir 4 may be made of water proof material and may be configured to hold liquid or paste-like medicament. The medicament's viscosity and active ingredients may vary from one to another. The medicament reservoir 4 may be sized to accommodate the volume of the medicament.

FIG. 2 is the top view of the second protective film layer 6. The second protective film layer 6 may be configured to cover the bottom of the adhesive pad 7. The shape of the second protective film layer 6 shown here is substantively oblong, but other shapes may be used. At one side of the second protective film layer 6 may be a removal handle 14, which is a portion of the second protective film layer 6 extending beyond the adhesive pad 7 upon assembling. A user can grab this removal handle 14 and peel to remove the second protective film layer 6 from the adhesive pad 7 prior to application of the electronic patch 1 to the skin. The second protective film layer 6 may protect the adhesive pad 7 from exposure to environmental factors while preventing medicament from seeping out of the medicament reservoir 4 before application to a user's skin. The second protective film layer 6 may be made from materials capable of preventing liquid from seeping through. By way of example, the second protective film layer 6 may be made from fluorosilicone coated polyester film, such as 3M Scotchpak Release Liner Fluorosilicone Coated Polyester Film 9709. Other suitable materials may be used for the second protective film layer 6.

FIG. 3 is the top view of the adhesive pad 7. The adhesive pad 7 may comprise a body with a hollow center in substantially oblong shape, even though other shapes are contemplated. The hollow center may substantially correspond to the shape of the first protective film layer 5. On one side of the adhesive pad 7 and in contact with the second protective film layer 6 may be adhesive tape with adhesive gel to adhere to the user's skin. By way of example, the adhesive tape may be commercially available polyurethane tapes, such as 3M CoTran Non-woven Polyurethane Tape, 9697. Other suitable materials may also be used to provide adhesion for the electronic patch 1.

FIG. 4 is the top view of the rate controlling membrane 13. Upon application of the electronic patch 1 to a user's skin, the rate controlling membrane 13 may sit between the medicament reservoir 4 and the user's skin. The rate controlling membrane 13 may be configured to allow passage of the medicament active ingredients from the reservoir 4 to the user's skin and a certain rate. The rate controlling membrane 13 may also be configured to respond to heat, such that the passage rate of the medicament through the rate controlling membrane 13 may change with the application of heat.

The first protective film layer 5 may be configured to isolate the medicament from the electronic controller 3. By way of example, the first protective film layer 5 may be made by fluoropolymer coated polyester film, such as 3M Scotchpak 1022 Release Liner Fluoropolymer Coated Polyester Film. Other suitable materials may also be used for the first protective film layer 5.

FIG. 5 is the top view of the reservoir retainer layer 15, which may be made of medical nonwoven fabric and present at the bottom of the medicament reservoir 4 and above the rate controlling membrane 13. The reservoir retainer layer 15 may line the bottom of the reservoir 4 and receive medicament while being in physical contact with the rate controlling membrane 13. During operation, the medicament may pass through the reservoir retainer layer 15 to the rate controlling membrane 13 and then to the user's skin.

In embodiments, the medicament reservoir 4 may be at the top of the medicament compartment. The medicament reservoir 4 may contain drug for permeation through the user's skin. The medicament reservoir 4 may be shaped and sized to fit with the first protective film layer 5 and generally to fit with other components of the medicament compartment 2. The medicament reservoir 4 may additionally be sized to accommodate the medicament to be stored in the medicament reservoir 4. The length of the medicament reservoir 4 may be, for example, 45-90 mm, while the width may be 30-80 mm. These dimensions are for illustrative purposes only and in no way shall limit the invention to these dimensions. Additionally, it is to be noted that the medicament compartment 2 in general and the medicament reservoir 4 in particular may be of other shapes apart from rectangular, including, but not limited to, for example, oblong, elliptical, circular, triangular, square, or other shapes. The bottom of the medicament reservoir 4 may be lined with a retainer layer made of medical nonwoven fabric, whereon the medicament may be deposited. Walls with uniform small thicknesses as compared to the length of the reservoir 4 may surround the perimeter and create the volume of the reservoir 4. The top of the medicament reservoir 4 may be lined with the first protective film layer 5.

In embodiments, the second protective film layer 6 may coat the adhesive pad 7, which may then be attached to the rate controlling membrane 3, which may then be attached to the medicament reservoir 4 at the reservoir retainer layer 15 and finally attached to the first protective film layer 5 to form the medicament compartment 2. Each medicament compartment 2 may carry an information storage means, which may be an Near Field Communication (NFC) chip, sticker, or tag, with identifying information and certain parameters concerning the medicament content, including amount, active ingredients, other ingredients, pharmaceutically acceptable excipients, safety information, contaminants, name of lab tester, date of testing, filing date, expiration date, user's name, and prescribing medical personnel, among other information. For botanical medicine such as medical cannabis, the information may further include name of the strain that was used to extract oil and/or cannabinoids, grower's name, extractor's name, growth time and season, among other information. The information storage means may be embedded inside in the medicament compartment 4 or attached to the packaging of the medicament compartment 2. Alternatively, a Quick Response (QR) code storing similar information may also be attached to the packaging of the medicament compartment 4. Information in the information storage means may be transmitted to an outside application or computer programming product operable on a computing article to be store, analyzed, and tracked for treatment purposes and/or research purposes.

FIG. 6 is the cross-sectional view of the electronic patch 1 taken along the X-X axis. At the bottom may be the second protective film layer 6, right above it may be the adhesive pad 7. The medicament reservoir 4 may be seen as the surrounding wall with a hollow space inside lined with a reservoir retainer layer 15, such as by medical nonwoven fabric at the bottom. At the bottom of the medicament reservoir 4 and underneath the reservoir retainer layer 15 may be the rate controlling membrane 13. At the top of the reservoir 4 and overarching it may be the electronic controller 3. The first protective film layer 5 may line the top of the medicament reservoir 4 and prevent medicament from seeping into the electronic controller 3.

In embodiments, the medicament contained in the medicament compartment 2 may be ready-made and filled during the fabrication of the medicament compartment 2. Alternatively, the medicament may be filled on-demand into pre-fabricated medicament compartments. Suitable pharmaceutical excipients may be chosen for each medicament. Where cannabinoids are the medicament, cannabinoids may be available as a natural component of cannabis oil or may be isolated, solid cannabinoids dissolved in a suitable liquid and mixed with suitable pharmaceutical excipients. Cannabinoids may be cannabidiol (CBD), tetrahydrocannabinol (THC), cannabinol (CBN), cannabigerol (CBG), or other cannabinoids.

In the electronic patch according to embodiments, situated above the medicament compartment 2 may be an electronic controller 3. The electronic controller 3 may operate to determine certain operating conditions, thereby affecting delivery rate or dosage, among other conditions. These effects are effectuated by controlling certain other parameters, such as temperature of the medicament during operation and heating duration of the electronic patch 1.

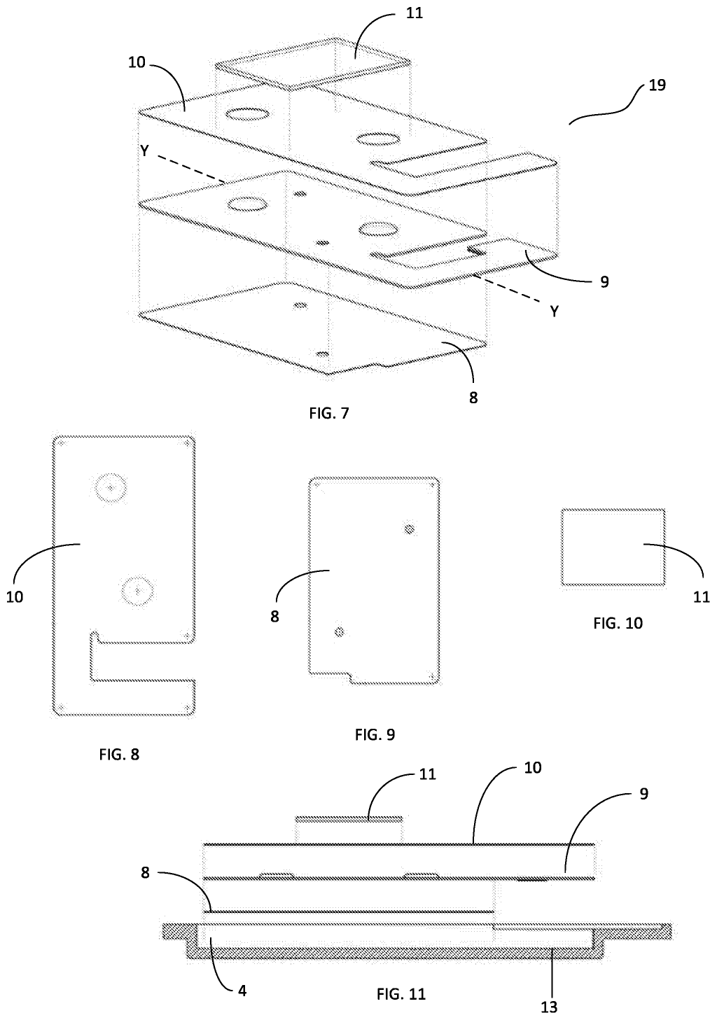

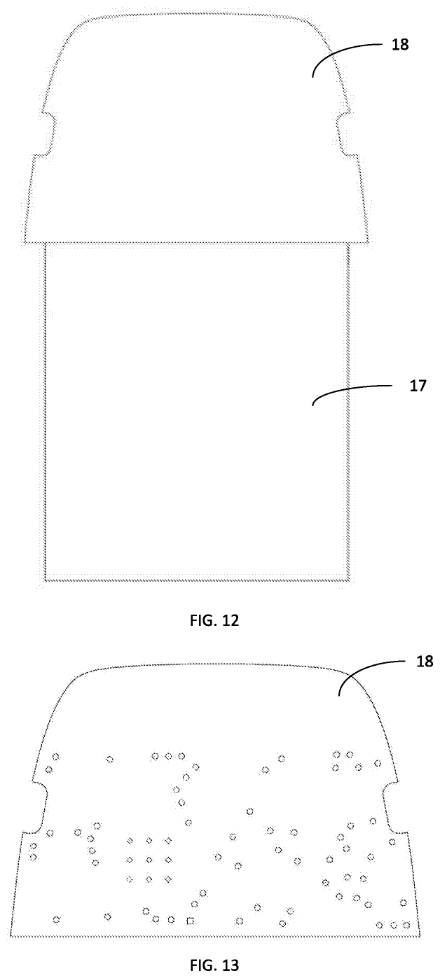

FIG. 7 is the exploded view of the different internal layers of the heating block 19 within the electronic controller 3. The electronic controller 3 may comprise a motherboard 18 (shown in FIG. 12) wherein electronic components for heating and operation may be housed, a battery, and a heating block. The heating block 19 (shown in FIG. 7) may comprise a thermal conductive layer 8 lining one side of the flexible circuit board 9, which may comprise a heating element and a thermistor, a thermal isolation layer 10 lining the other side of the flexible circuit board 9, and a foam tape 11 to provide cushion between the internal layers of the heating block 19 and other parts within the electronic controller 3. The thermal conductive layer 8 may conduct heat from the heating element on the flexible circuit board 9 to the medicament, while the thermal isolation layer 10 may prevent heat from escaping the heating element on the other side.

In embodiments, situated above the flexible circuit board 9 may be the thermal isolation layer 10. FIG. 8 is the top view of the thermal isolation layer 10. The thermal isolation layer 10 may prevent heat from escaping from the heating block 19 components to surrounding components, including the electronic controller housing 12. The thermal isolation layer 10 may operate to prevent overheating of other components as well as preserving heat such that it can be conducted through the thermal conductive layer 8. The thermal isolation layer 10 may be made of materials suitable for efficient heat isolation while light enough to fit with the electronic controller's size, such as polyimide or polyimide film. By way of example, DuPont Kapton polyimide films may be used. Other materials with low thermal conductivity appropriate for the purpose may also be used. The thermal isolation layer 10 may be sized to fit with the top of the flexible circuit board 9, such that it can effectively prevent heat from escaping from the heating element.

FIG. 9 is the top view of the thermal conductive layer 8. At the bottom and in contact with the medicament reservoir 4 may be the thermal conductive layer 8. During operation, the motherboard 18 may regulate electricity supplied by the battery 17. Electrical current from the battery 17 as regulated by the motherboard 19 may be distributed to the copper conductive lining present on the flexible circuit board 9. The copper conductive lining may act as the heating element and conduct heat to thermal conductive layer 8, which in turn may conduct heat to the medicament reservoir 4 and the medicament. The thermal conductive layer 8 may be made of materials suitable for transmitting heat, such as ceramic with heat conductive properties. Other materials with heat conductive properties appropriate for this purpose may also be used. The thermal conductive layer 8 may be sized to fit with the top of the medicament reservoir 4.

In embodiments, situated directly above the thermal conductive layer 8 may be the flexible circuit board 9, which is a sheet with flexible printed circuit having a heating element, which may be copper conductive lining, and a thermistor. During operation, the flexible printed circuit 9 may receive electrical current from the battery 17 as regulated by the motherboard 18 to heat up the heating element, which is the copper conductive lining. Heat from the heating element may be conducted to the thermal conductive layer 8, which in turn may be conducted to the medicament within the medicament reservoir 4 during operation of the electronic patch 1 through contact between the thermal conductive layer 8 and the medicament in the reservoir 4. The flexible circuit board 9 may be shaped such that upon assembly into the electronic patch 1, some portions of the motherboard 18 are not in contact with the flexible circuit board 9, thereby prevent these portions from heating up and preserving the integrity of electronic components within the motherboard 18. In FIG. 7, the flexible circuit board 9 is shown as having a gap near one end. This gap may correspond to the space above where electronic components soldered onto the motherboard 18 may sit. A rechargeable battery 17 (shown in FIG. 12) may be present to provide energy for the operation of the electronic patch 1. One-time use batteries may also be used.

FIG. 10 is the top view of a foam tape according to embodiments. In embodiments, the electronic patch 1 may further comprise a foam tape 11, which may serve as a cushion between the top of the thermal isolation layer 10 and the motherboard 18 and/or the battery 17. The foam tape 11 may be sized to provide adequate cushioning and may also provide a non-conductive adhesive means. In embodiments, the foam tape 11 may comprise two adhesive layers. Generally, the foam tape 11 may be smaller in dimension than the layers underneath it within the electronic controller 3, even though other dimension suitable for the purpose may be considered.

FIG. 11 is the cross sectional, exploded view of medicament compartment 2 and the heating block within the electronic controller 3 taken along the Y-Y axis. At the top may be the foam tape 11, which may be attached to the thermal isolation layer 10 at a location to provide a cushion between the thermal isolation layer 10 and the electronic controller housing 12. The thermal isolation layer 10 may be situated on top of the flexible circuit board 9, such that the upper surface area of the flexible circuit board 9 is completely covered and isolated by the thermal isolation layer 10. Underneath the flexible circuit board 9 may be the thermal conductive layer 8, which may conduct heat to the medicament. Upon assembled, the thermal conductive layer 8 may fit on top of the medicament reservoir 4, such that it comes into physical contact with the medicament contained therein. At the bottom of the medicament reservoir 4 may be the rate controlling membrane 13. The rate controlling membrane 13 may touch the user's skin upon application of the electronic patch 1 to the skin.

FIG. 12 is the top view of the motherboard 18 and the battery 17. In embodiments, the motherboard 18 and the battery 17 may be assembled such that they form one layer and sit on top of the heating block 19, above the thermal isolation layer 10 with the foam tape 11 between the motherboard 18/battery assembly 17 and the thermal isolation layer 10. The motherboard 18 may house electronic components responsible for operation of the electronic patch. A LED light may be present on the motherboard 18, which may lit up to indicate that the electronic patch 18 is in operation.

FIG. 13 is the top view of the motherboard 18 with drill holes. The drill holes may indicate where electronic components may be soldered in. As shown herein, the drills are mostly concentrated at one end of the motherboard 18 while the opposite end has no drills. Other arrangement of drill holes for different electrical component arrangements may be used.

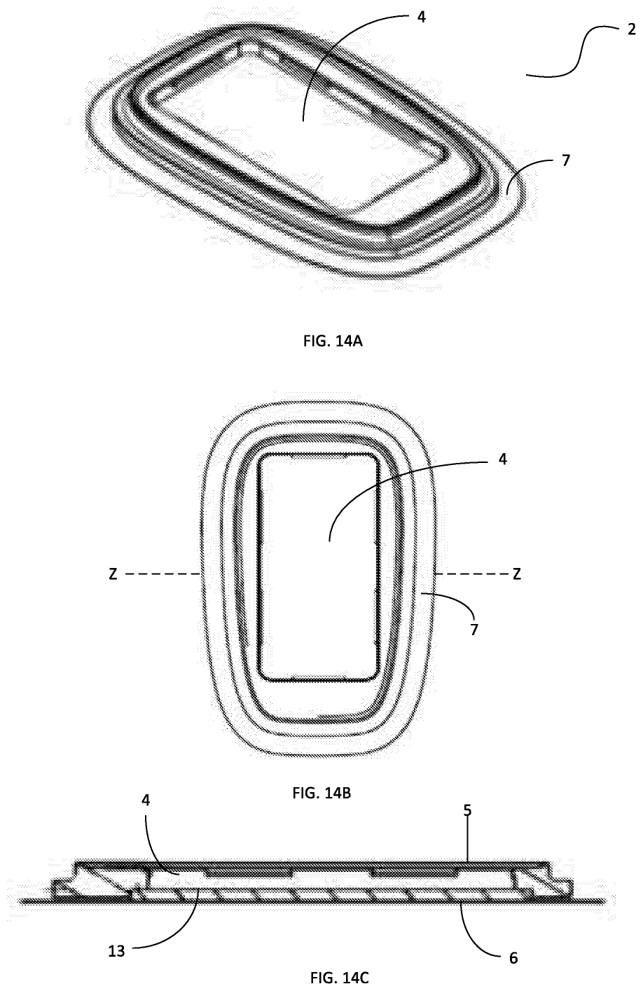

FIG. 14A is a perspective view of the medicament compartment 2. The reservoir 4 may be situated in the middle of the medicament compartment 2. In this embodiment as shown here, the medicament reservoir 4 is of substantial rectangular shape with round edges and a small thickness. It is contemplated that the medicament reservoir 4 may have different shapes and different dimensions to accommodate the medicament amount and type as well as other configurations of the electronic patch 1. The adhesive pad 7 may be of larger dimensions than the medicament reservoir 4, such that the adhesive pad 7 creates a surface that encloses the surface of the rate controlling membrane 13 and the medicament reservoir 4.

FIG. 14B is the top view of the medicament compartment 2, with the reservoir 4 shown in substantially rectangular shape and the adhesive pad 7 in substantially oblong shape, and the adhesive pad's surface area expanding beyond the reservoir's surface area.

FIG. 14C is the cross section view of the medicament compartment 2 taken along the Z-Z line. The second protective film layer 6 may be at the bottom of the medicament compartment 2, sealing the rate controlling membrane 13. The medicament reservoir 4 may be above the rate controlling membrane 13, while the first protective film layer 5 may be at the top of the reservoir 4.

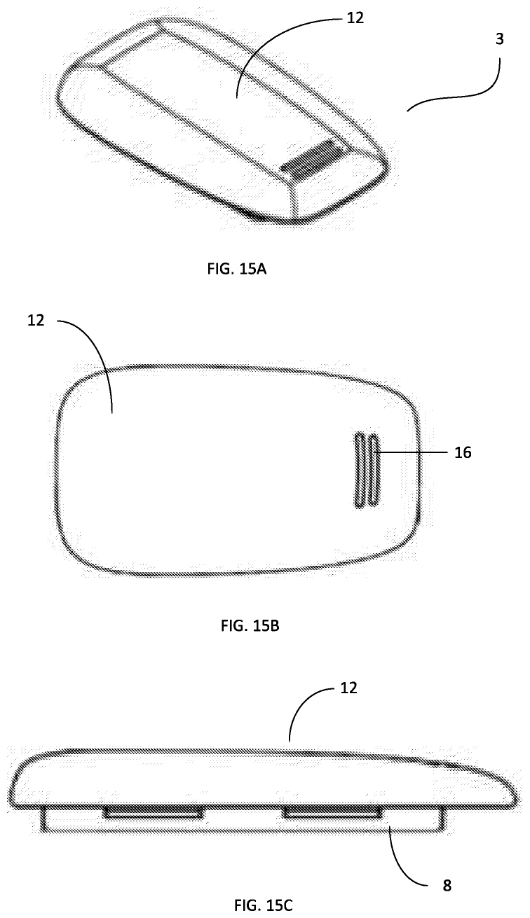

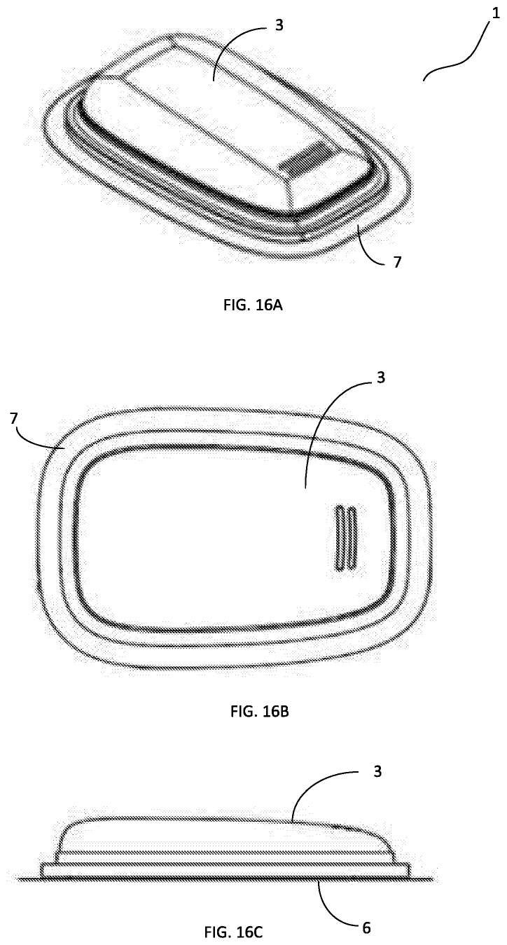

FIG. 15A is a perspective view of the electronic controller 3 as fully assembled inside the housing 12. In embodiments, the thermal conductive layer 8, the flexible circuit board 9, the thermal isolation layer 10, the foam tape 11, the motherboard 18, and the battery 17 may be housed in a housing 12 to comprise the electronic controller 3. The housing 12 may have an opening 16 for the LED display to be visible to the user. The housing 12 may be in an oblong shape as shown in the drawings included herein, but may be in any other shape. The housing 12 may be sized to fit with the medicament compartment 2 such that the electronic patch 1 as a whole has an aesthetic appeal.

FIG. 15B is the top view of the electronic controller 3 and FIG. 15C is the right side view of the electronic controller 3. In FIG. 15B an opening 16 is shown on the body of the electronic controller housing 12. This opening 16 is through which a LED light may be seen, indicating the operation status of the electronic patch 1. In FIG. 15C, the thermal conductive layer 8 may be seen at the bottom of the electronic controller 3.

FIG. 16A is a perspective view of the electronic patch 1 fully assembled. The electronic controller 3 may be fitted on top of the medicament compartment 2 to form a complete electronic patch 1. As shown here, the electronic patch 1 has a generally rectangular shape with a thickness that is smaller than the length. The electronic patch 1 may be fashioned in different shapes and sizes to provide suitable delivery methods for specific medicaments.

FIG. 16B is the top view of the complete electronic patch 1, while FIG. 16C is the right side view of the same. In FIG. 16C, the second protective film layer 6 may be seen at the bottom of the electronic patch. The medicament compartment 2 and the electronic controller 3 may be attached to each other by an attachment means such as a snap-fit. They may also be attached to each other by gel. These attachment mechanisms allow for removal and replacement of the medicament compartment 2. Alternatively, the medicament compartment 2 and the electronic controller 3 may be welded or molded together during production. In this case, they may not be separated for replacement of the medicament compartment 2.

In embodiments, the electronic patch 1 may further comprise a Light Emitting Diode (LED), which may turn on when the electronic patch 1 is in operation. The LED display may be visible on the housing of the electronic controller 3 through the LED opening 16 to give indication of the operation status of the electronic patch 1.

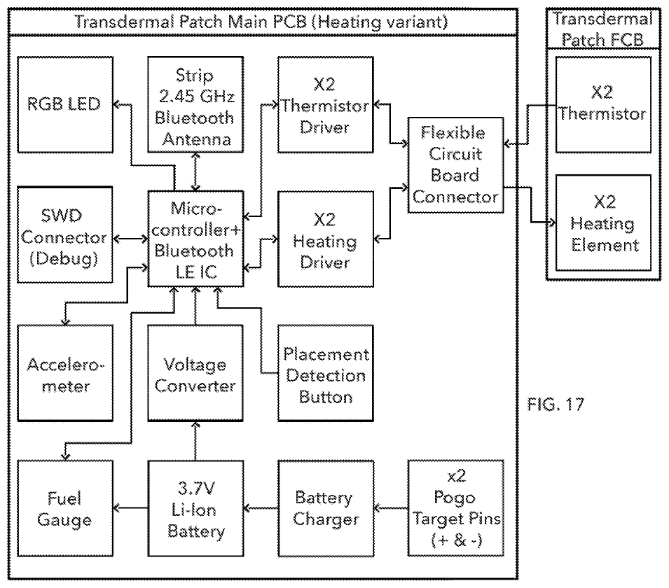

FIG. 17 illustrates the operating layout of the motherboard 18 and the flexible circuit board 9. The motherboard 18 is a printed circuit board where electronic components may be operationally connected and physically attached for mechanical stability. The flexible circuit board (FCB) 9 may host the thermistor and the heating element. The FCB 9 may be connected to the PCB 18 by a flexible circuit board connector, which is a wired connection.

On the main PCB, the center of the operation may be the microcontroller and the Bluetooth Low Energy (LE) integrated circuit (IC). The heating cycle may start with the Bluetooth antenna receiving commands from a remote application, then the microcontroller receiving signals from the Bluetooth antenna and sending signal to the heating driver. Through the FCB connector, the heating element may receive energy from the battery and heat up according to signal received. The thermistor, in communication with the microcontroller via the thermistor driver and the FCB connector, may communicate the change in temperature and thus the temperature is “read” and ultimately collected as data.

In embodiments, the motherboard 18 may be powered by a chargeable battery. The motherboard 18 may have Pogo target pins connected to a battery charger, which may charge a battery. For example, the battery may be a 3.7 volt lithium-ion battery, but other types and capacities of the battery are contemplated. Current generated from the battery may pass through a voltage converter to adapt to the need of the microcontroller and/or Bluetooth low energy integrated circuit. Energy from the battery may also pass through a fuel gauge to the microcontroller to output state of charge and battery status for the user to see in the control application and/or computer programming product (software) interface.

Data received from an outside computing processor through commands received from an application and/or a computer programming product may be communicated from the Bluetooth within the outside computing processor to a Bluetooth antenna in the electronic controller, for example but not limited to, through a Strip 2.45 GHz Bluetooth antenna, which may communicate back to the microcontroller. The microcontroller, upon receiving the commands, may activate the heating driver and/or thermistor driver.

In embodiments, the electronic controller may comprise a Red-Green-Blue (RGB) Light Emitting Diode (LED) display, which may receive signals from the microcontroller and output light to signal whether the electronic patch is operating. When the remote application or computer programming product sends signal to the electronic controller to start heating, the microcontroller may also send signal to the RGB LED to output light to visually indicate that the electronic patch is heating.

In embodiments, an accelerometer may be present to detect the movement of the user while wearing the electronic patch. This information may be communicated to the microcontroller and used as part of the controlling scheme for the electronic patch. For example, the electronic patch may be programmed such that, upon resting by the user for a predetermined length of time, the electronic patch would activate to release the medicament. In another example, when the accelerometer detects additional movement by the user, it may activate heating to provide the user with needed medication due to increased movement.

In embodiments, a Serial Wire Debug (SWD) may be connected to the microcontroller to enable debugging and resolving problems. Debugging and resolving problems may be necessary where operational issues are detected.

In embodiments, a placement detection button may be present as part of the controlling mechanism to optimize the electronic patch's operation. The placement detection button may detect, for example, the location on the user's body where the electronic patch is attached. Attachment to different locations may require different rate of medicament delivery. For example, when the electronic patch is attached to the thigh, the placement detection button may detect this and communicate this information to the microcontroller within the electronic controller. The electronic controller may use this information, together with other parameters, to adapt the heating temperature and intervals to optimize medicament delivery to the user.

In embodiments, the electronic patch may further comprise a computer programming product operable on a remote computing article, such as a software or application on a smart phone, a tablet, a desktop, or a laptop. The computer programming product may be configured to receive information from the electronic patch, send wireless signals to the electronic patch, and control the operation of the electronic patch. The computer programming product may also be configured to collect, store and aggregate data from the electronic patch, as well as self-learn from the data collected through machine learning.

In operation of the electronic patch, upon attaching the medicament compartment to the electronic controller, the electronic patch is complete and may be attached to the user's skin for medicine delivery. A remote computing program product such as an application may receive information available on the information storage means attached to the medicament compartment. Retrieval of this information may be by scanning of the information storage means using a scanner, such as a mobile device scanner. The electronic controller may be set to certain operating parameters, such as temperature to exert onto the medicament reservoir, length of heating intervals, and length of non-heating intervals, among other parameters, using the remote computing program product. Variations in operating parameters may enable the control of medicine delivery rate to the user. The electronic patch may then heat up and medicament may cross the rate controlling membrane to reach the user's skin and then cross the user's skin. The heating cycle may be terminated and restart by operating parameters pre-set based on the information available in the information storage means or as pre-set by the user. Operating parameters may be collected by the electronic controller and communicated to another, remote computing processor or processors. Such data may be collected and stored for analysis for each individual user, such that medical professionals caring for the user may use this data as part of their care decision.

In embodiments, the medicament compartment may be produced separately from the electronic controller and may be produced by three-dimensional printing. The medicament included in the medicament compartment may vary and may be filled per a prescription to a specific user. Different active ingredients, different pharmaceutically acceptable excipients, and different dosage may be filled into each medicament compartment. Such data may be stored in an information storage means, such as a Near Field Communication (NFC) chip, sticker, or tag with uniquely identifying parameters, thereby enabling control of the delivery and data collection. The information storage means may be present on the medicament compartment or on the packaging of the medicament compartment to interact with the electronic controller. Information may also be stored in a QR code present on the packaging of the medicament compartment.

In embodiments, the medicament compartment may be produced at a provider's site, such as at a pharmacy according to a prescription, and may have different information embedded in an NFC chip, sticker, or tag, or a QR code, which may communicate with the electronic controller. The medicament compartment may be disposable, such that after each use it may be removed from the electronic controller and disposed of, while the electronic controller may be re-used with another medicament compartment. In this instance, the new medicament compartment may have a different electronic information storage means storing different information. Alternatively, the medicament compartment may be re-filled with similar medicament or with a different medicament and may be re-attached to the electronic controller for further use.

In embodiments, during operation, the LED display may turn on to indicate the operation status of the electronic patch. Electrical current generated by the battery during operation may power the LED display, which serves as indicator that the electronic patch is in operation.

In embodiments, control of the electronic patch may be through a remote computing means, such as by an application or a computer programming product on a computing article, the application or computer programming product may have an interface displayable and interacting with users on a computing article. Such computing articles may be a mobile computing device, a mobile phone, a tablet, a desktop, a laptop, a computer embedded into another device, among other computing articles. The remote computing article may communicate with the electronic controller by a wired connection or wirelessly. Different computing articles having the application or the computer programming product operating and collecting data from electronic patches according to embodiments herein may communicate with each other to exchange and aggregate collected data for further use, such as to conduct research and studies.

The electronic patch and a computing program product such as a mobile application operable on a separate computing article may be provided together as a system for users. The computing program product may be configured to receive or retrieve information stored on the information storage means available on the medicament compartment. The computing program product may be configured for machine learning, such that data collected during operation of the electronic patch may be used to teach the computing program product specific knowledge for operational decision as tailored for the individual using the electronic patch. The computing program products operating various electronic patches may also be configured to communicate with each other and exchange data. Data collected and exchanged may be used as the input for machine learning for each electronic patch according to embodiments.

In embodiments, the electronic patch may deliver medicament, in particular, cannabinoids, such that plasma concentration of cannabinoid reaches around 10-50 ng/ml. Typical cannabinoid plasma concentrations after topical application are below 10 ng/ml. This compares with 100 ng/ml of cannabinoid plasma concentration by inhalation—the cannabinoid delivery route with the highest plasma concentration peak. Use of the electronic patch as described herein thus increases medicament delivery rate from a simple patch.

The parts herein may be made by methods known in the art, such as injection molding or relatively precise machining. The housing may be made from plastic with heat tolerance. The motherboard may be made from a printed circuit board with electronic components physically and electrically connected to the connections present on the printed circuit board. The medicament compartment may be made of plastic that is heat durable and water proof. Suitable materials may be selected to suit the purpose of each components within the electronic patch.

All references, including publications, patent applications, and patents cited herein are hereby incorporated by reference to the same extent as if each reference were individually and specifically indicated to be incorporated by reference and were set forth in its entirety herein.

It will be readily apparent to those skilled in the art that a number of modifications and changes may be made without departing from the spirit and the scope of the present invention. It is to be understood that any ranges, ratios, and range of ratios that can be derived from any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art will appreciate that such values are unambiguously derivative from the data presented herein.

What is claimed is:

1. An electronic patch for transdermal delivery of medicament, comprising:
a medicament compartment to contain a medicament, comprising:
a reservoir configured to hold a medicament, the reservoir having on one end a first protective film layer and on the other end a rate controlling membrane, the first protective film payer and the rate controlling membrane situated approximately opposing each other and separated by the height of the reservoir;
an adhesive pad attached to the reservoir at the end where the rate controlling membrane resides, the adhesive pad being in contact with the reservoir at the perimeter of the reservoir; and
a second protective film layer in physical contact with the adhesive pad on the opposite side from the rate controlling membrane;
an electronic controller in contact with and operatively connected to the reservoir, the electronic controller comprising:
a thermal conductive layer configured to conduct thermal energy to the medicament compartment, the thermal conductive layer being in physical contact with and operatively connected to the first protective film layer;
a flexible circuit board having a heating element and a thermistor;
a motherboard having electronic circuits and electronic components attached to the motherboard, the motherboard operatively connected to the flexible circuit board; and
a thermal isolation layer situated between the flexible circuit board and the motherboard, the thermal isolation layer configured to substantially reduce thermal energy conduction from the flexible circuit board;
a battery operatively connected to the motherboard; and
a housing to house the electronic controller components;
wherein the motherboard comprises a microcontroller, and
wherein the medicament compartment and the electronic controller are operatively connected to each other.

2. The electronic patch of claim 1, wherein the electronic controller is configured to collect data from the electronic patch.

3. The electronic patch of claim 2, wherein the data collected comprise temperature during heating episodes of the medicament compartment, length of the heating episodes, frequency of the heating episodes, and types of medicament.

4. The electronic patch of claim 1, wherein the electronic controller is configured to wirelessly communicate with another computing processor.

5. The electronic patch of claim 1, wherein the medicament compartment further comprises an electronic information storage means having information concerning the medicament contained inside.

6. The electronic patch of claim 1, wherein the electronic controller is configured to wirelessly receive commands from a computing processor and execute the commands received.

7. The electronic patch of claim 1, wherein the medicament compartment is capable of being removed from electronic controller and replaced by another, similar medicament compartment.

8. The electronic patch of claim 1, wherein the medicament compartment is produced by three-dimensional printing.

9. The electronic patch of claim 1, wherein the electronic controller is configured to transmit thermal energy to the medicament compartment in intervals.

10. The electronic patch of claim 1, further comprising a medicament residing inside the reservoir.

11. The electronic patch of claim 10, further comprising a foam tape situated at the top of the thermal isolation layer.

12. The electronic patch of claim 11, further comprising a Light Emitting Diode display to indicate operating status of the electronic patch.

13. The electronic patch of claim 12, further comprising a computer programming product operable on a remote computing article and providing an electronic control interface capable of interacting with the electronic patch.

14. The electronic patch of claim 13, wherein the computer programming product is configured to send operating commands to the electronic patch, receiving data from the electronic patch, storing and aggregating received data, and conducting machine learning based in the received data.

15. The electronic patch of claim 13, further comprising an accelerometer configured to detect movement of the user and communicate collected data to the motherboard, wherein the motherboard uses collected data to adjust the electronic patch's operation.

16. The electronic patch of claim 15, further comprising a placement detection button to detect the body part where the electronic patch is placed, wherein the detection button is configured to communicate this information to the motherboard and wherein the motherboard uses collected data to adjust the electronic transdermal patch's operation.

17. A method to deliver medicine transdermally, comprising the steps of:
placing the electronic patch on a user's skin at an administration site, the electronic patch comprising:
a medicament compartment to contain a medicament, comprising:
a reservoir configured to hold a medicament, the reservoir having on one end a first protective film layer and on the other end a rate controlling membrane, the first protective film payer and the rate controlling membrane situated approximately opposing each other and separated by the height of the reservoir;
an adhesive pad attached to the reservoir at the end where the rate controlling membrane resides, the adhesive pad being in contact with the reservoir at the perimeter of the reservoir; and
a second protective film layer in physical contact with the adhesive pad on the opposite side from the rate controlling membrane;
an electronic controller in contact with and operatively connected to the reservoir, the electronic controller comprising:
a thermal conductive layer configured to conduct thermal energy to the medicament compartment, the thermal conductive layer being in physical contact with and operatively connected to the first protective film layer;
a flexible circuit board having a heating element and a thermistor;
a motherboard having electronic circuits and electronic components attached to the motherboard, the motherboard operatively connected to the flexible circuit board;
a thermal isolation layer situated between the flexible circuit board and the motherboard, the thermal isolation layer configured to substantially reduce thermal energy conduction from the flexible circuit board;
a foam tape situated at the top of the thermal isolation layer;
a battery operatively connected to the motherboard;
a housing to house the electronic controller components;
a medicament residing inside the reservoir; and
a computer programming product operable on a remote computing article and providing an electronic control interface capable of interacting with the electronic patch;
wherein the motherboard comprises a microcontroller,
wherein the medicament compartment and the electronic controller are operatively connected to each other, and
wherein the computer programming product is configured to send operating commands to the electronic patch, receiving data from the electronic patch, storing and aggregating received data, and conducting machine learning based in the received data;
opening the electronic control interface on the remote computing article;
setting operating parameters for the electronic transdermal patch; and
starting the operating procedure for the electronic transdermal patch.

18. The method of claim 17, wherein the operating parameters are temperature, duration of heating cycles, and frequency of heating cycles.

19. The method of claim 17, further comprising the steps of:
collecting data from the electronic patch's operation;
transmitting collected data to a remote computing processor;
storing collected data in a computing article; and
analyzing stored data to make a medical care decision.

20. A method to conduct medical studies, comprising the steps of:
placing the electronic patch on a user's skin at an administration site, the electronic patch comprising:
a medicament compartment to contain a medicament, comprising:
a reservoir configured to hold a medicament, the reservoir having on one end a first protective film layer and on the other end a rate controlling membrane, the first protective film payer and the rate controlling membrane situated approximately opposing each other and separated by the height of the reservoir;
an adhesive pad attached to the reservoir at the end where the rate controlling membrane resides, the adhesive pad being in contact with the reservoir at the perimeter of the reservoir; and
a second protective film layer in physical contact with the adhesive pad on the opposite side from the rate controlling membrane;
an electronic controller in contact with and operatively connected to the reservoir, the electronic controller comprising:
a thermal conductive layer configured to conduct thermal energy to the medicament compartment, the thermal conductive layer being in physical contact with and operatively connected to the first protective film layer;
a flexible circuit board having a heating element and a thermistor;
a motherboard having electronic circuits and electronic components attached to the motherboard, the motherboard operatively connected to the flexible circuit board;
a thermal isolation layer situated between the flexible circuit board and the motherboard, the thermal isolation layer configured to substantially reduce thermal energy conduction from the flexible circuit board;
a foam tape situated at the top of the thermal isolation layer;
a battery operatively connected to the motherboard;
a housing to house the electronic controller components;
a medicament residing inside the reservoir; and
a computer programming product operable on a remote computing article and providing an electronic control interface capable of interacting with the electronic patch;
wherein the motherboard comprises a microcontroller,
wherein the medicament compartment and the electronic controller are operatively connected to each other, and
wherein the computer programming product is configured to send operating commands to the electronic patch, receiving data from the electronic patch, storing and aggregating received data, and conducting machine learning based in the received data;
opening the electronic control interface on the remote computing processor;
setting operating parameters for the electronic transdermal patch;
starting the operating procedure for the electronic transdermal patch;
collecting data from the heating cycles;
transmitting collected data to a remote computing processor;
storing collected data in a computing article;
repeating the above steps with various electronic patches and various users for a finite time;
analyzing stored data to make medical care decision; and
aggregating data from different users and conducting research and study based on the aggregated data.
